(19) United States
(12) Patent Application Publication
(10) Pub. No.: US 2013/0059793 A1
(43) Pub. Date: Mar. 7, 2013

(54) EGF RECEPTOR MIMICKING PEPTIDES

(71) Applicants: Marina Cardó-Vila, Houston, TX (US); Ricardo J. Giordano, Sao Paulo (BR); John Mendelsohn, Houston, TX (US); Wadih Arap, Houston, TX (US); Renata Pasqualini, Houston, TX (US)

(72) Inventors: Marina Cardó-Vila, Houston, TX (US); Ricardo J. Giordano, Sao Paulo (BR); John Mendelsohn, Houston, TX (US); Wadih Arap, Houston, TX (US); Renata Pasqualini, Houston, TX (US)

(73) Assignee: BOARD OF REGENTS, THE UNIVERSITY OF TEXAS SYSTEM, Austin, TX (US)

(21) Appl. No.: 13/577,809

(22) PCT Filed: Feb. 8, 2011
(86) PCT No.: PCT/US2011/024073

§ 371 Date: Nov. 21, 2012

Related U.S. Application Data
(60) Provisional application No. 61/302,405, filed on Feb. 8, 2010.

(51) Int. Cl. A61K 38/16; C07K 19/00; C12N 15/63; C12P 21/02; A61K 38/10; G01N 21/75; C07K 7/08; C07K 7/06; A61K 38/08; C07K 14/00; C12N 15/11

(52) U.S. Cl. 514/19.3; 530/324; 530/387.3; 530/322; 530/350; 530/351; 435/320.1; 435/69.1; 536/23.1; 435/7.1; 530/325; 530/326; 530/327; 530/328; 530/329; 530/330

(57) ABSTRACT

Provided are peptides which can mimic the epidermal growth factor receptor (EGFR), e.g., by selectively binding TGF-α and/or EGF. In certain embodiments, the peptides are retro-inverted peptides. The peptides may be used as soluble decoys for TGF-α and/or EGF, and anti-cancer properties of peptides are demonstrated both in vitro and in vivo. The peptides may be administered alone or comprised in a fusion construct, imaging construct, and/or a therapeutic construct, e.g., for the treatment of a cancer.

This application claims priority to U.S. Application No. 61/302,405 filed on Feb. 8, 2010, the entire disclosure of which is specifically incorporated herein by reference in its entirety without disclaimer.

### BACKGROUND OF THE INVENTION

1. Field of the Invention

The present invention relates generally to the fields of molecular biology and medicine. More particularly, it concerns EGF receptor (EGFR) mimicking peptides.

2. Description of Related Art

The epidermal growth factor receptor (EGFR) is a member of the ErbB family of tyrosine kinase receptors (Gusterson and Hunter, 2009; Baselga, 2006). Several lines of evidence indicate that the EGFR is abnormally activated in many types of epithelial tumors. The first therapeutic agent targeted to the EGFR is a monoclonal antibody, cetuximab, which blocks ligand-binding and thus inhibits tyrosine kinase activity (Kawamoto et al., 1983). In the past few years, it has become clear that specific somatic EGFR mutations present in non-small cell lung cancer potentiate responses to certain low molecular weight tyrosine kinase inhibitors and monoclonal antibodies (Gusterson and Hunter, 2009; Lynch et al., 2004; Mellinghoff et al., 2005; Paez et al., 2004; Sharma et al., 2007; Scott et al., 2007); mutation of the K-ras gene also has been associated with survival in patients with advanced colon cancer treated with cetuximab (Karapetis et al., 2008). These agents, both antibodies and tyrosine kinase inhibitors, prevent ligand-induced receptor activation and downstream signaling, and result in cell cycle arrest, promotion of apoptosis, and inhibition of angiogenesis (Mendelsohn and Baselga, 2006; Dassonville et al., 2007).

There are three general classes of agents that can inhibit tyrosine kinase receptors: blocking antibodies, small kinase inhibitors, and soluble ligand traps or receptor decoys. However, only agents belonging to the first two classes are currently available for therapeutic intervention: monoclonal antibodies directed at the ligand-binding extracellular domain of the receptor (e.g., cetuximab, panitumumab, zalutumumab, nimotuzumab, and matuzumab), and low-molecular weight inhibitors of intracellular tyrosine kinase activity (e.g., gefitinib, erlotinib, and lapatinib). Currently, only a few EGFR molecular decoys have been identified, such as Argos, which is a 419 residue protein identified in Drosophila which can act as an antagonist of EGFR signaling by binding EGF (Klein et al., 2004; Klein et al., 2008), and a recombinant form of the extracellular domain of ErbB4 that antagonizes ligand-induced receptor tyrosine phosphorylation (Gilmore and Riese, 2004). Unfortunately and as stated above, no EGFR decoy has yet been developed that is available for clinical therapeutic use. Given the significance of EGFR in cancer and the very limited number of identified EGFR molecular receptor decoys, there is a clear need for the development of new EGFR molecular decoys.

### SUMMARY OF THE INVENTION

The present invention overcomes limitations in the prior art by providing new EGFR molecular decoys which can act as soluble ligand traps. For example, peptides including _{D}(CARVC) (SEQ ID NO:1) are provided and can bind the EGFR ligands EGF and TGF-α and inhibit tumor cell proliferation in vitro and in vivo. In certain embodiments, the identified peptides can provide a significant therapeutic advantage due to the short size of the peptides, cyclization, and/or the use of D-amino acids to resist enzymatic breakdown in the body, thus extending the therapeutic half-life of the molecules.

As shown in the below examples, _{D}(CARVC) can inhibit tumor cell proliferation in vitro, in cells, and in vivo, and experimental evidence indicates that this new class of small drug candidates may function through an EGFR-decoy mechanism. In contrast to other EGFR-targeting agents such as cetuximab, this ligand-sequestering drug still may be active and may be used as a candidate for translation in the setting of downstream K-ras gene mutations. This may be particularly useful considering that human tumors containing KRAS mutations often express high levels of ErbB ligands (Dlugosz et al., 1995; Baba et al., 2000; Sweet-Cordero et al., 2004). It has also been shown that KRAS mutations are not sufficient to confer resistance to EGFR inhibition (Fujimoto et al., 2005). Combinatorial peptide library selection involving key receptor-ligand tumor pathways was used to identify additional molecules which may function as soluble ligand traps for EGFR, including those listed in Table 1. It is anticipated that these peptides may be used to treat essentially any disease which is characterized by an increase in EGFR function or which would therapeutically benefit from a decrease in TGF-α or EGF signaling.

An aspect of the present invention relates to a peptide comprising _{D}(ARV) or VRA, wherein the peptide is 50 or less amino acids in length, and wherein the peptide can selectively bind epidermal growth factor (EGF) or transforming growth factor alpha (TGF-α). In certain embodiments, the peptide is 15 or less or 10 or less amino acids in length. The peptide may be a cyclic peptide. The peptide may comprise _{D}(CARVC) or CVRAC (SEQ ID NO:2). The peptide may selectively bind epidermal growth factor (EGF) and transforming growth factor alpha (TGF-α). In certain embodiments, the peptide comprises _{D}(CARVC), and the peptide is 7 or less amino acids in length. The peptide may consist of _{D}(CARVC). The peptide may be 7 amino acids or less in length. The peptide may be comprised in a pharmaceutically acceptable formulation. The peptide may comprise CVRAC.

The peptide may be conjugated or fused to a second agent such as a polypeptide, or a therapeutic or diagnostic agent. The peptide may be prepared by a process comprising obtaining a nucleic acid coding region the encodes the peptide and fusing said coding region in frame to a nucleic acid coding region for the polypeptide to form a fused coding region, and expressing said fused coding regions to provide the peptide fused with said polypeptide. The second agent may be a therapeutic agent, further defined as a drug, a chemotherapeutic agent, a radioisotope, a pro-apoptosis agent, an anti-angiogenic agent, a hormone, a cytokine, a cytotoxic agent, a cytocidal agent, a cytostatic agent, a peptide, a protein, an antibiotic, an antibody, a Fab fragment of an antibody, a hormone antagonist, a nucleic acid or an antigen. The second agent may be an anti-angiogenic agent selected from the group consisting of thrombospondin, angiostatin, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4, and minocycline. The second agent may be a pro-apoptosis agent selected from the group consisting of etoposide, ceramide sphingomyelin, Bax, Bid, Bik, Bad, caspase-3, caspase-8, caspase-9, fas, fas ligand, fadd, fap-1, tradd, faf, rip, reaper, apoptin, interleukin-2 converting enzyme or annexin V. The second agent may be a cytokine selected from the group consisting of interleukin 1 (IL-1), IL-2, IL-5, IL-10, IL-12, IL-18, interferon-γ(IF-γ), IF α, IF-β, tumor necrosis factor-α (TNF-α), or GM-CSF (granulocyte macrophage colony stimulating factor). The second agent may be a molecular complex, such as a virus, a bacteriophage, a bacterium, a liposome, a microparticle, a magnetic bead, a yeast cell, a mammalian cell or a cell. The virus may be chosen from the group consisting of adenovirus, retrovirus adeno-associated virus (AAV), and AAVP. In various embodiments, the virus may be further defined as containing a gene therapy vector. The peptide may be attached to a eukaryotic expression vector, such as a gene therapy vector.

In various embodiments, the second agent may be a diagnostic agent, such as an imaging agent. The imaging agent may comprise chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) erbium (III), lanthanum (III), gold (III), lead (II), or bismuth (III). The agent may comprise a radioisotope, such as astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine ¹²³ iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} or yttrium⁹⁰. The peptide may be comprised in a pharmaceutically acceptable composition.

Another aspect of the present invention relates to a method of making a polypeptide comprising obtaining a nucleic acid coding region that encodes the peptide and fusing said coding region in frame to a nucleic acid coding region for the polypeptide to form a fused coding region, and expressing said fused coding regions to provide the peptide fused with said polypeptide.

Yet another aspect of the present invention relates to a nucleic acid that encodes a protein or peptide comprising _{D}(ARV) or VRA; wherein the peptide is 10 or less amino acids in length. The peptide may comprise _{D}(CARVC) or CVRAC. The nucleic acid may be operably linked to a heterologous promoter.

Another aspect of the present invention relates to a method of treating a hyperproliferative disease such as cancer comprising administering to a subject an EGFR-mimicking peptide of the present invention. The cancer may be selected from the group consisting of lung cancer, gastrointestinal cancer, colon cancer, anal cancer, and glioblastoma multiforme. The subject may be a mammal, such as a human. The peptide may be administered in a pharmaceutically acceptable carrier. The method may further comprise administering a second therapeutic agent to the subject.

Yet another aspect of the present invention relates to a method for imaging cells expressing epidermal growth factor (EGF) or transforming growth factor alpha (TGF-α) comprising exposing cells to an EGFR-mimicking peptide conjugated or fused to an imaging agent. The cells may comprise cancer cells, including but not limited to lung cancer cells, gastrointestinal cancer cells, anal cancer cells, or glioblastoma multiforme cells.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D: Screening of a combinatorial random peptide library on EGFR ligands EGF, TGFα, and cetuximab. (FIG. 1A) EGF panning VEGF and BSA were used as negative control proteins in (FIG. 1A) and (FIG. 1B). (FIG. 1B) TGFα panning (FIG. 1C) M225 monoclonal antibody (the original murine version of cetuximab) was immobilized onto microtiter wells at a concentration of 2 μg. The CX₇C phage library was incubated with each of the target proteins. Shown are the relative TU obtained from each well coated with M225, mIgG, or BSA after three rounds of selection (RI-RIII). (FIG. 1D) Specificity of the peptides recovered from RIII targeting M225 was recapitulated upon binding to cetuximab on a fourth round of selection (RIV). Results are expressed as mean±standard error of the mean (SEM) of triplicate wells.

FIGS. 2A-C: Mapping candidate epitopes within the EGFR. (FIG. 2A) Amino acid sequence corresponding to the extracellular domain of the EGFR (accession # NP_{—}005219). Leu1 is the first residue after the signal peptide. Arrowhead designates the signal peptide cleavage site. Yellow highlights indicate five consensus regions to which peptides derived from library screenings (on the ligands EGF, TGFα, and cetuximab) were clustered. Green and red boxes pinpoint the reciprocal residues in the two EGFR molecules involved in dimerization. (FIG. 2B) Location of a cetuximab-binding region within the EGFR structure. Light green and light red ribbons indicate the backbone of each EGFR homodimer. Purple designates the TGFα ligand bound to the EGFR. Insert: red and green indicate residues involved in EGFR dimerization (see FIG. 2A). Yellow ribbon shows the location of CVRAC within the EGFR homodimer (residues 283-287). (FIG. 2C) CVRAC-displaying phage binds specifically to cetuximab, EGF, and TGF-α. VEGF or BSA served as negative controls for binding. Recombinant proteins were coated onto microtiter wells at 10 μg/ml, and wells were incubated with either CVRAC-phage or CVAAC-phage (SEQ ID NO:7) (alanine scanning control). An insertless phage was an additional negative control. Phage input was 10⁹ TU per well. Results are expressed as mean±SEM of triplicate wells.

FIGS. 3A-D: Molecular interaction of CVRAC, cetuximab and EGFR. (FIG. 3A) Synthetic peptides (CVRACGAD (SEQ ID NO:3) or CVRAC), compared to an unrelated control peptide (SDNRYIGSW (SEQ ID NO:4)), specifically bind to cetuximab. BSA served as an additional negative control, and the EGFR, as a positive control. (FIG. 3B) Concentration-dependent inhibition of binding of cetuximab to the EGFR by the synthetic peptides CVRACGAD and CVRAC, in comparison to negative controls: an EGFR sequence-derived peptide (CQKCDPSC (SEQ ID NO:5)) and an unrelated negative control peptide. (FIG. 3C) Phage displaying alanine scanning versions of the CVRAC peptide (CARAC (SEQ ID NO:6) and CVAAC) were used to identify critical residues based on their capacity to bind to cetuximab. Insertless phage served as a negative control. (FIG. 3D) Polyclonal antibody against CVRAC recognized the EGFR. Bars represent mean±SEM.

FIGS. 4A-E: The retro-inverso peptidomimetic of the CVRAC motif is recognized by cetuximab and inhibits binding of cetuximab to the EGFR. (FIG. 4A) Human HN5 tumor cells were treated with increasing concentrations of cetuximab (black line). Cells were also exposed to either 60 μM (red line) or 180 μM (blue line) CVRAC. Unrelated control peptide (purple line) or EGFR-related control peptide (green line) had no effect on cetuximab activity. A representative experiment is depicted. Experiments were repeated four times with similar results. Bars represent mean±SEM. (FIG. 4B) Binding of retro-inverso D-form peptides (plated at 10 μg/ml) to cetuximab in an ELISA-based assay. Equivalent amounts of IgGs (cetuximab, anti-CVRAC, or h-IgG) were analyzed for binding to CVRAC or to its retro-inverso peptidomimetic _{D}(CARVC). (FIG. 4C) Effect of the synthetic peptides on HN5 tumor cells. Cells were incubated with increasing concentrations (up to 250 mM) of the peptide CVRAC, the retro-inverso peptidomimetic _{D}(CARVC), or a negative control peptide. Viability in the absence of peptide was set to 100%. (FIG. 4D) Inhibition of EGFR:cetuximab association, monitored by SPR in the presence of synthetic peptides or peptidomimetic _{D}(CARVC). Bars represent mean±SEM. (FIG. 4E) Analysis of receptor autophosphorylation in cells stimulated with EGF or control media for 15 min, after which cetuximab or synthetic peptides were added with the growth factor to evaluate inhibition. Receptors were immunoprecipitated with antibodies against phosphorylated (p) pEGFR and were immunoblotted with anti-phosphotyrosine IgG. This representative experiment shows that _{D}(CARVC) specifically inhibits the phosphorylation of the EGFR in human HN5 tumor cells.

FIGS. 5A-B: CVRAC-targeted phage homes to tumor. (FIG. 5A) Targeting tumors versus control organs. Phage displaying the peptide CVRAC or CVAAC, or insertless negative control phage, were administered intravenously into mice bearing EF43.fgf-4-derived tumors. An anti-phage antibody was used for staining H&E staining, with the corresponding fluorescence-based immunostaining, are shown in tumors (FIG. 5A). Tumor-bearing mice received CVRAC phage, CVAAC phage, or insertless control phage as indicated. Cohorts of tumor-bearing mice (n=5 mice/group) were used. A representative experiment is shown. Scale bar, 100 μm. (FIG. 5B) Treatment of tumor-bearing mice with peptides and peptidomimetics. Balb/c mice bearing EF43.fgf-4-derived tumors were divided into size-matched cohorts (n=7 mice/group); individual tumor volumes are represented before (black circles) and after (white circles). Peptides and peptidomimetics were administered at 750 μg/mouse/dose for 5 days. Shown are mean tumor volumes±SEM.

FIGS. 6A-B: The prototype peptidomimetic drug _{D}(CARVC) functions through an EGFR-decoy mechanism. (FIG. 6A)_{D}(CARVC) displaces EGF from the EGFR. The EGFR was coated onto 96-well plates at decreasing concentrations. Increasing molar concentrations of the synthetic peptidomimetic _{D}(CARVC) were used to evaluate competitive inhibition of EGF binding (squares). _{D}(CAAVC (SEQ ID NO:8)) was used as a negative peptidomimetic control at the same concentrations (circles). Cetuximab (12 nM) served as a positive control for the displacement of EGF from the EGFR. (FIG. 6B)_{D}(CARVC) displaces the binding of TGFα from the EGFR. Evaluation of the competitive inhibition of the binding TGFα to the EGFR by increasing molar concentrations (as indicated) of the synthetic peptidomimetic _{D}(CARVC). Bars represent mean±SEM.

FIGS. 7A-C: Inhibition of binding of cetuximab to the EGFR by a panel of synthetic peptides. Peptides selected from the consensus motifs in all three EGFR ligands were tested for binding inhibition of cetuximab to EGFR.

FIG. 8: Retro-inverso peptidomimetic design. Schematic representation of the retro-inverso peptidomimetic and minimal energy structure of CVRAC and _{D}(CARVC) is shown. Residues are color-coded: cysteine (Cys, orange), alanine (Ala, red), arginine (Arg, green) and valine (Val, blue). Dotted areas indicate amino acid side chains.

FIGS. 9A-C: Inhibition of binding of cetuximab to the EGFR on different tumor cell lines. Cells: (FIG. 9A) HN5, (FIG. 9B) GEO, and (FIG. 9C) EF43.fgf-4 were exposed to increasing concentrations of the drug _{D}(CARVC) (black line) or of the control peptidomimetic _{D}(CAAVC) (blue line). Experiments were repeated four times with similar results. A representative experiment is shown. Bars represent mean±SEM.

FIGS. 10A-B: CVRAC-targeted phage homes to tumor. An anti-phage antibody was used for staining H&E staining, with the corresponding fluorescence-based immunostaining (FIG. 10A) brain and (FIG. 10B) kidney were used as negative control organs.

### DESCRIPTION OF ILLUSTRATIVE EMBODIMENTS

The present invention overcomes limitations in the prior art by providing peptides which can mimic the epidermal growth factor receptor (EGFR) by binding the EGFR ligands EGF and/or TGF-α. In various embodiments, these EGFR mimicking peptides may be used therapeutically to treat a cancer.

EGFR is a tyrosine kinase which is central to human tumorigenesis. Typically, three classes of drugs inhibit tyrosine kinase pathways: blocking antibodies, small kinase inhibitors, and soluble ligand receptor traps/decoys. Only the first two types of EGFR-binding inhibitory drugs are clinically available; notably, no EGFR-decoy has yet been developed for therapeutic use. To address this need, provided herein are small molecules mimicking EGFR which can functionally behave as soluble decoys for EGF and TGFα, ligands that would otherwise activate downstream signaling. As shown in the below examples, after combinatorial library selection on EGFR ligands, a panel of binding-peptides was narrowed by structure-function analysis (see, e.g., Table 1). The most active motif was CVRAC (EGFR 283-287), which is necessary-and-sufficient for specific EGFR ligand binding. The synthetic retro-inverted derivative, _{D}(CARVC), was further investigated, and anti-tumor properties were demonstrated both in vitro and in vivo.

### EGFR Mimicking Peptides

EGFR mimicking peptides were identified by the following general approach. The inventors designed and utilized an in tandem approach that comprises mapping of interactive sites on EGFR ligands, followed by the chemical generation and evaluation of derivative consensus motif analogues. A combinatorial library screening was first performed in representative EGFR ligands in vitro to select and identify a panel of consensus motifs. Solid-phase synthesis was subsequently used to produce pertinent peptides and peptidomimetic drug candidates (see, e.g., Table 1). The EGFR drug decoy candidate _{D}(CARVC), a synthetic, low-molecular weight, retro-inverted, water-soluble peptidomimetic, was evaluated by in vitro, in cellulo, and in vivo assays and demonstrated significant anti-tumor activity. Aside from the retro-inversion approach, which generates degradation-resistant D-peptidomimetics (Meister, 1965), cyclization was also used in an attempt to improve the bioavailability of the peptide. Various EGFR mimicking peptides herein, such as _{D}(CARVC), can act as a structural and functional drug decoy of this tyrosine kinase receptor with tumor targeting attributes and may be used for translational applications. Select synthetic peptides which can bind EGF and/or TGF-α are presented below in Table 1.

TABLE 1
Synthetic EGFR-mimicking peptides selected from overlapping consensus
motifs.

| | | | EGFR Homology |
|---|---|---|---|
| Synthetic Peptides | Structure | Selected on | Region |
| QRNYDLSFL | Linear | EGF & TGFα | ⁴⁷Q-L⁵⁵ |
| (SEQ ID NO: 9) | | | |
| CQKCDPSC | Cyclic | Cetuximab & TGFα | ¹⁶³C-C¹⁷⁰ |
| (SEQ ID NO: 10) | | | |
| PNGSCW | Linear | Cetuximab | ¹⁷¹P-W¹⁷⁶ |
| (SEQ ID NO: 11) | | | |
| AQQCSGRCRGKSPSD | Cyclic | EGF & TGFα | ¹⁹¹A-D²⁰⁷ |
| (SEQ ID NO: 12) | | | |
| CRKFRDEATC | Cyclic | All EGFR ligands | ²²⁷C-C²³⁶ |
| (SEQ ID NO: 13) | | | |
| CKDTC | Cyclic | All EGFR ligands | ²³⁵C-C²⁴⁰ |
| (SEQ ID NO: 14) | | | |
| CVRACGAD | Cyclic | All EGFR ligands | ²⁸³C-C²⁹⁰ |
| (SEQ ID NO: 15) | | | |
| THTPPLDPQEL | Linear | EGF & TGFα | ³⁵8T-L³⁶⁸ |
| (SEQ ID NO: 16) | | | |
| IIRGRTK | Linear | EGF & TGFα | ⁴⁰¹I-K⁴⁰⁷ |
| (SEQ ID NO: 17) | | | |
| CSPEGC | Cyclic | All EGFR ligands | ⁴⁸⁶C-C⁴⁹¹ |
| (SEQ ID NO: 18) | | | |
| CLPQAMNIT | Linear | Cetuximab | ⁵³⁸C-T⁵⁴⁶ |
| (SEQ ID NO: 19) | | | |
| CTGRGPDNCIQ | Cyclic | All EGFR ligands | ⁵⁴⁷C-Q⁵⁵⁷ |
| (SEQ ID NO: 20) | | | |
| IQCAHYIDGPHC | Cyclic | All EGFR ligands | ⁵⁵⁶I-C⁵⁶⁷ |
| (SEQ ID NO: 21) | | | |
| CPAGVM | Linear | Cetuximab | ⁵⁷¹C-M⁵⁷⁶ |
| (SEQ ID NO: 22) | | | |
| CTGPGLEGCPTNGPK | Cyclic | All EGFR ligands | ⁶⁰⁴C-K⁶¹⁸ |
| (SEQ ID NO: 23) | | | |

### Retro-Inverted EGFR-Mimicking Peptides

In various aspects, EGFR mimicking peptides may be synthesized as retro-inverted peptides, e.g., to avoid degradation and/or improve the half-life of a peptide. For example, as shown in the below examples, _{D}(CARVC) can target EGF and TGF-β, and it may be used, e.g., to treat a cancer in vivo. It is anticipated that a retro-inverted peptide may be generated for essentially any of the EGFR-mimicking peptides presented herein while retaining many, substantially, or essentially all of the pharmacological actions of the EGFR-mimicking peptide. In certain embodiments, an EGFR-mimicking retro-inverted peptide may be 3, 4, 5, 6, 7, 8, 9, 10, 11, 12, 13, 14, or 15 amino acids long, and in certain embodiments 5-10 amino acids long. Retro-inverted peptides may be synthetically produced by known methods including solid-phase synthesis.

Peptide-based drugs are often susceptible to degradation by proteolytic enzymes; consequently, the biological activity of a peptide depends directly on its stability in serum. Retro-inverted peptide modification (i.e., reversal of the direction of the primary peptide sequence plus inversion of the chirality of each individual residue to the D-enantiomer) of biologically active motifs has been shown to increase the stability of peptidomimetic drug candidates (Chorev and Goodman, 1993), because most natural mammalian proteases do not cleave D-residues their non-peptide bonds (Meister, 1965). In general, this retro-inversion approach can result in peptidomimetics with strong topological correlation to the parent peptide because the resulting side-chain disposition is similar (i.e., the positions of side-chains are preserved) but carbonyl and amide groups are inter-converted (i.e., the positions of carbonyl and amino groups in the backbone of the peptide are exchanged).

Tumor Targeting with EGFR-Mimicking Peptides

In certain embodiments a EGFR-mimicking peptide, such as _{D}(CARVC) or CVRAC, may be conjugated to an imaging or cytotoxic agent and used for tumor targeting. As shown in the below examples _{D}(CARVC) or CVRAC selectively accumulate at or in tumors. Without wishing to be bound by any theory, these agents may home to an EGFR “ligand-rich” tumor microenvironment, such as those with high local concentrations of the native ligands EGF and/or TGFα. As described in further detail below, various imaging agents and/or cytotoxic moieties may be chemically conjugated or covalently bonded to an EGFR-mimicking peptide.

### Proteins and Peptides

In certain embodiments, the present invention concerns compositions comprising at least one EGFR-mimicking peptide. In certain embodiments, the size of an EGFR-mimicking peptide may comprise, but is not limited to, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues. In various embodiments, an EGFR mimicking peptide may be from 3 to 25 amino acids in length, from 3 to 15 amino acids in length, or from 3 to 10 amino acids in length. It will be generally appreciated that, in certain embodiments, smaller EGFR-mimicking peptides may have certain advantages, including for example a decreased cost associated with synthesis. An EGFR mimicking peptide may, in certain embodiments, be chemically conjugated or covalently bonded to a second peptide or protein, such as a cytotoxic protein or a protein which may be utilized in imaging.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid, including but not limited to those shown on Table 2 below.

TABLE 2
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

Proteins or peptides may be made by any technique known to those of skill in the art, including the expression of proteins, polypeptides, or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (www.ncbi.nlm.nih.gov). The coding regions for known genes may be amplified and/or expressed using the techniques disclosed herein or as would be know to those of ordinary skill in the art. Alternatively, various commercial preparations of proteins, polypeptides and peptides are known to those of skill in the art.

### Peptide Mimetics

Another embodiment for the preparation of polypeptides according to the invention is the use of peptide mimetics. Mimetics are peptide-containing molecules that mimic elements of protein secondary structure. See, for example, Johnson et al. (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

### Fusion Proteins

Other embodiments of the present invention concern fusion proteins. These molecules generally have all or a substantial portion of a targeting peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the instant invention comprise a targeting peptide linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually and protein or peptide could be incorporated into a fusion protein comprising a targeting peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

### Protein Purification

In certain embodiments a protein or peptide may be isolated or purified. In one embodiment, these proteins may be used to generate antibodies for tagging with any of the illustrated barcodes (e.g. polymeric Raman label). Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, HPLC (high performance liquid chromatography) FPLC (AP Biotech), polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. One of the more efficient methods of purifying peptides is fast performance liquid chromatography (AKTA FPLC) or even A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

### Imaging Agents and Radioisotopes

In certain embodiments, an EGFR mimicking peptide may be attached to an imaging agent of use for imaging and diagnosis of various diseased organs, tissues or cell types. For example, a prostate cancer selective targeting peptide may be attached to an imaging agent, provided to a subject and the precise boundaries of the cancer tissue may be determined by standard imaging techniques, such as CT scanning, MRI, PET scanning, etc. Alternatively, the presence or absence and location in the body of metastatic prostate cancer may be determined by imaging using one or more targeting peptides that are selective for metastatic prostate cancer. Targeting peptides that bind to normal as well as cancerous prostate tissues may still be of use, as such peptides would not be expected to be selectively localized anywhere besides the prostate in disease-free individuals. Naturally, the distribution of a prostate or prostate cancer selective targeting peptide may be compared to the distribution of one or more non-selective peptides to provide even greater discrimination for detection and/or localization of diseased tissues.

Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), and gadolinium may be particularly useful in certain embodiments. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled proteins or peptides of the present invention may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides according to the invention may be labeled with technetium ^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups that are often used to bind radioisotopes that exist as metallic ions to peptides are diethylenetriaminepenta-acetic acid (DTPA) and ethylene diaminetetra-acetic acid (EDTA). Also contemplated for use are fluorescent labels, including rhodamine, fluorescein isothiocyanate and renographin.

In certain embodiments, the claimed proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

### Synthetic Peptides

Because of their relatively small size, the targeting peptides of the invention can be synthesized in solution or on a solid support in accordance with conventional techniques. Various automatic synthesizers are commercially available and can be used in accordance with known protocols. See, for example, Stewart and Young, 1984; Tam et al., 1983; Merrifield, 1986; and Barany and Merrifield, 1979, each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

### Antibodies

In certain embodiments, it may be desirable to make antibodies against the identified targeting peptides or their receptors. The appropriate targeting peptide or receptor, or portions thereof, may be coupled, bonded, bound, conjugated, or chemically-linked to one or more agents via linkers, polylinkers, or derivatized amino acids. This may be performed such that a bispecific or multivalent composition or vaccine is produced. It is further envisioned that the methods used in the preparation of these compositions are familiar to those of skill in the art and should be suitable for administration to humans, i.e., pharmaceutically acceptable. Preferred agents are the carriers are keyhole limpet hemocyanin (KLH) or bovine serum albumin (BSA).

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Harlow and Lane, 1988; incorporated herein by reference).

In various embodiments of the invention, circulating antibodies from one or more individuals with a disease state may be obtained and screened against phage display libraries. Targeting peptides that bind to the circulating antibodies may act as mimeotopes of a native antigen, such as a receptor protein located on an endothelial cell surface of a target tissue. For example, circulating antibodies in an individual with prostate cancer may bind to antigens specifically or selectively localized in prostate tumors. As discussed in more detail below, targeting peptides against such antibodies may be identified by phage display. Such targeting peptides may be used to identify the native antigen recognized by the antibodies, for example by using known techniques such as immunoaffinity purification, Western blotting, electrophoresis followed by band excision and protein/peptide sequencing and/or computerized homology searches. The skilled artisan will realize that antibodies against disease specific or selective antigens may be of use for various applications, such as detection, diagnosis and/or prognosis of a disease state, imaging of diseased tissues and/or targeted delivery of therapeutic agents.

### Cross-Linkers

The EGFR mimicking peptides may be attached to surfaces or to therapeutic agents and other molecules using a variety of known cross-linking agents. Methods for covalent or non-covalent attachment of proteins or peptides are well known in the art. Such methods may include, but are not limited to, use of chemical cross-linkers, photoactivated cross-linkers and/or bifunctional cross-linking reagents. Exemplary methods for cross-linking molecules are disclosed in U.S. Pat. Nos. 5,603,872 and 5,401,511, incorporated herein by reference. Non-limiting examples of cross-linking reagents of potential use include glutaraldehyde, bifunctional oxirane, ethylene glycol diglycidyl ether, carbodiimides such as 1-ethyl-3-(3-dimethylaminopropyl) carbodiimide or dicyclohexylcarbodiimide, bisimidates, dinitrobenzene, N-hydroxysuccinimide ester of suberic acid, disuccinimidyl tartarate, dimethyl-3,3′-dithio-bispropionimidate, azidoglyoxal, N-succinimidyl-3-(2-pyridyldithio)propionate and 4-(bromoadminoethyl)-2-nitrophenylazide.

Homobifunctional reagents that carry two identical functional groups are highly efficient in inducing cross-linking. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied.

In certain embodiments, it may be appropriate to link one or more targeting peptides to a liposome or other membrane-bounded particle. For example, targeting peptides cross-linked to liposomes, microspheres or other such devices may be used to deliver larger volumes of a therapeutic agent to a target organ, tissue or cell type. Various ligands can be covalently bound to liposomal surfaces through the cross-linking of amine residues. Liposomes containing phosphatidylethanolamine (PE) may be prepared by established procedures. The inclusion of PE provides an active functional amine residue on the liposomal surface.

In another non-limiting example, heterobifunctional cross-linking reagents and methods of use are disclosed in U.S. Pat. No. 5,889,155, incorporated herein by reference. The cross-linking reagents combine a nucleophilic hydrazide residue with an electrophilic maleimide residue, allowing coupling in one example, of aldehydes to free thiols. The cross-linking reagent can be modified to cross-link various functional groups.

Other techniques of general use for proteins or peptides that are known in the art have not been specifically disclosed herein, but may be used in the practice of the claimed subject matter.

### Nucleic Acids

In certain embodiments, nucleic acids may encode a targeting peptide, a receptor protein, a fusion protein or other protein or peptide. The nucleic acid may be derived from genomic DNA, complementary DNA (cDNA) or synthetic DNA. Where incorporation into an expression vector is desired, the nucleic acid may also comprise a natural intron or an intron derived from another gene. Such engineered molecules are sometime referred to as “mini-genes.” In various embodiments of the invention, targeting peptides may be incorporated into gene therapy vectors via nucleic acids.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of almost any size, determined in part by the length of the encoded protein or peptide.

It is contemplated that targeting peptides, fusion proteins and receptors may be encoded by any nucleic acid sequence that encodes the appropriate amino acid sequence. The design and production of nucleic acids encoding a desired amino acid sequence is well known to those of skill in the art, using standardized codon tables. In preferred embodiments, the codons selected for encoding each amino acid may be modified to optimize expression of the nucleic acid in the host cell of interest. Codon preferences for various species of host cell are well known in the art.

In addition to nucleic acids encoding the desired peptide or protein, the present invention encompasses complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

Nucleic acids for use in the disclosed methods and compositions may be produced by any method known in the art, such as chemical synthesis (e.g. Applied Biosystems Model 3900, Foster City, Calif.), purchase from commercial sources (e.g. Midland Certified Reagents, Midland, Tex.) and/or standard gene cloning methods. A number of nucleic acid vectors, such as expression vectors and/or gene therapy vectors, may be commercially obtained (e.g., American Type Culture Collection, Rockville, Md.; Promega Corp., Madison, Wis.; Stratagene, La Jolla, Calif.).

### Vectors for Cloning, Gene Transfer and Expression

In certain embodiments expression vectors are employed to express the targeting peptide or fusion protein, which can then be purified and used. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are known.

### Regulatory Elements

The terms “expression construct” or “expression vector” are meant to include any type of genetic construct containing a nucleic acid coding for a gene product in which part or all of the nucleic acid coding sequence is capable of being transcribed. In preferred embodiments, the nucleic acid encoding a gene product is under transcriptional control of a promoter. A “promoter” refers to a DNA sequence recognized by the synthetic machinery of the cell, or introduced synthetic machinery, required for initiating the specific transcription of a gene. The phrase “under transcriptional control” means that the promoter is in the correct location and orientation in relation to the nucleic acid to control RNA polymerase initiation and expression of the gene.

In various embodiments, the human cytomegalovirus (CMV) immediate early gene promoter, the SV40 early promoter, the Rouse sarcoma virus long terminal repeat, rat insulin promoter, and glyceraldehyde-3-phosphate dehydrogenase promoter can be used to obtain high-level expression of the coding sequence of interest. The use of other viral or mammalian cellular or bacterial phage promoters that are known in the art to achieve expression of a coding sequence of interest is contemplated as well, provided that the levels of expression are sufficient for a given purpose.

Where a cDNA insert is employed, one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

### Selectable Markers

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin, and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

### Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome, and express viral genes stably and efficiently have made them attractive candidates for the transfer of foreign genes into mammalian cells (Ridgeway, 1988; Nicolas and Rubinstein, 1988; Baichwal and Sugden, 1986; Temin, 1986). Preferred gene therapy vectors are generally viral vectors.

In using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

DNA viruses used as gene vectors include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986).

An exemplary method for in vivo delivery involves the use of an adenovirus expression vector. Although adenovirus vectors have a low capacity for integration into genomic DNA, this feature is counterbalanced by the high efficiency of gene transfer afforded by these vectors. “Adenovirus expression vector” is meant to include, but is not limited to, constructs containing adenovirus sequences sufficient to (a) support packaging of the construct and (b) to express an antisense or a sense polynucleotide that has been cloned therein.

Generation and propagation of adenovirus vectors that are replication deficient depend on a helper cell line, such as the 293 cell line, which was transformed from human embryonic kidney cells by Ad5 DNA fragments and constitutively expresses E1 proteins (Graham et al., 1977). Since the E3 region is dispensable from the adenovirus genome (Jones and Shenk, 1978), adenovirus vectors, with the help of 293 cells, carry foreign DNA in either the E1, the E3, or both regions (Graham and Prevec, 1991).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. Racher et al. (1995) disclosed methods for culturing 293 cells and propagating adenovirus.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). In some embodiments, gene therapy vectors are based upon adeno-associated virus (AAV).

Other gene transfer vectors may be constructed from retroviruses. (Coffin, 1990.) The retroviral genome contains three genes, gag, pol, and env. that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences, and also are required for integration in the host cell genome (Coffin, 1990). In various embodiments, a lentiviral vector may be used to deliver an expression vector.

In order to construct a retroviral vector, a nucleic acid encoding protein of interest is inserted into the viral genome in the place of certain viral sequences to produce a virus that is replication-defective. In order to produce virions, a packaging cell line containing the gag, pol, and env genes, but without the LTR and packaging components, is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., 1975).

Other viral vectors may be employed as expression constructs. Vectors derived from viruses such as vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984), and herpes viruses may be employed. They offer several attractive features for various mammalian cells (Friedmann, 1989; Ridgeway, 1988; Baichwal and Sugden, 1986; Coupar et al., 1988; Horwich et al., 1990).

Several non-viral methods for the transfer of expression constructs into cultured mammalian cells also are contemplated. These include calcium phosphate precipitation (Graham and van der Eb, 1973; Chen and Okayama, 1987; Rippe et al., 1990; DEAE dextran (Gopal, et al. 1985), electroporation (Tur-Kaspa et al., 1986; Potter et al., 1984), direct microinjection, DNA-loaded liposomes and lipofectamine-DNA complexes, cell sonication, gene bombardment using high velocity microprojectiles, and receptor-mediated transfection (Wu and Wu, 1987; Wu and Wu, 1988). Some of these techniques may be successfully adapted for in vivo or ex vivo use.

In a further embodiment of the invention, the expression construct may be entrapped in a liposome. Liposome-mediated nucleic acid delivery and expression of foreign DNA in vitro has been very successful. Wong et al. (1980) demonstrated the feasibility of liposome-mediated delivery and expression of foreign DNA in cultured chick embryo, HeLa, and hepatoma cells. Nicolau et al. (1987) accomplished successful liposome-mediated gene transfer in rats after intravenous injection.

### Pharmaceutical Compositions

In certain embodiments, a EFGR mimicking peptide may be included or administered in a pharmaceutical composition. Where clinical applications are contemplated, it may be necessary to prepare pharmaceutical compositions—expression vectors, virus stocks, proteins, antibodies and drugs—in a form appropriate for the intended application. Generally, this will entail preparing compositions that are free or essentially free of impurities that could be harmful to humans or animals.

One generally will desire to employ appropriate salts and buffers to render delivery vectors stable and allow for uptake by target cells. Aqueous compositions of the present invention may comprise an effective amount of a protein, peptide, fusion protein, recombinant phage and/or expression vector, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the proteins or peptides of the present invention, its use in therapeutic compositions is contemplated. Supplementary active ingredients also can be incorporated into the compositions.

The active compositions of the present invention may include classic pharmaceutical preparations. Administration of these compositions according to the present invention are via any common route so long as the target tissue is available via that route. This includes oral, nasal, buccal, rectal, vaginal or topical. Alternatively, administration may be by orthotopic, intradermal, subcutaneous, intramuscular, intraperitoneal, intraarterial or intravenous injection. Such compositions normally would be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

### Therapeutic Agents

In certain embodiments, therapeutic agents may be attached to a EGFR mimicking peptide for selective delivery to, for example, a non-metastatic or metastatic cancer. Agents or factors suitable for use may include any chemical compound that induces apoptosis, cell death, cell stasis and/or anti-angiogenesis or otherwise affects the survival and/or growth rate of a cancer cell.

### Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Tsujimoto et al., 1985). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

Non-limiting examples of pro-apoptosis agents contemplated within the scope of the present invention include gramicidin, magainin, mellitin, defensin, cecropin, (KLAKLAK)₂ (SEQ ID NO:24).

### Angiogenic Inhibitors

In certain embodiments the present invention may concern administration of targeting peptides attached to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline.

Proliferation of tumors cells relies heavily on extensive tumor vascularization, which accompanies cancer progression. Thus, inhibition of new blood vessel formation with anti-angiogenic agents and targeted destruction of existing blood vessels have been introduced as an effective and relatively non-toxic approach to tumor treatment. (Arap et al., 1998a; 1998b; Ellerby et al., 1999). A variety of anti-angiogenic agents and/or blood vessel inhibitors are known. (e.g., Folkman, 1997; Eliceiri and Cheresh, 2001).

### Cytotoxic Agents

A wide variety of anticancer agents are well known in the art and any such agent may be coupled to a cancer targeting peptide for use within the scope of the present invention. Exemplary cancer chemotherapeutic (cytotoxic) agents of potential use include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and “Remington: The Science and Practice of Pharmacy,” 20th edition, Gennaro, Lippincott, 2000, each incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Of course, all of these dosages and agents described herein are exemplary rather than limiting, and other doses or agents may be used by a skilled artisan for a specific patient or application. Any dosage in-between these points, or range derivable therein is also expected to be of use in the invention.

### Alkylating Agents

Alkylating agents are drugs that directly interact with genomic DNA to prevent cells from proliferating. This category of chemotherapeutic drugs represents agents that affect all phases of the cell cycle, that is, they are not phase-specific. An alkylating agent, may include, but is not limited to, nitrogen mustard, ethylenimene, methylmelamine, alkyl sulfonate, nitrosourea or triazines. They include but are not limited to: busulfan, chlorambucil, cisplatin, cyclophosphamide (cytoxan), dacarbazine, ifosfamide, mechlorethamine (mustargen), and melphalan.

### Antimetabolites

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

### Natural Products

Natural products generally refer to compounds originally isolated from a natural source (e.g., a herbal composition), and identified as having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

### Antibiotics

Certain antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of cytotoxic antibiotics include, but are not limited to, bleomycin, dactinomycin, daunorubicin, doxorubicin (Adriamycin), plicamycin (mithramycin) and idarubicin.

### Miscellaneous Agents

Miscellaneous cytotoxic agents that do not fall into the previous categories include, but are not limited to, platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are not limiting and it is contemplated that any known cytotoxic, cytostatic or cytocidal agent may be attached to targeting peptides and administered to a targeted organ, tissue or cell type within the scope of the invention.

### Cytokines and Chemokines

In certain embodiments, it may be desirable to couple specific bioactive agents to one or more targeting peptides for targeted delivery to an organ, tissue or cell type. Such agents include, but are not limited to, cytokines and/or chemokines.

The term “cytokine” is a generic term for proteins released by one cell population that act on another cell as intercellular mediators. Examples of cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha. and -beta; mullerian-inhibiting substance; mouse gonadotropin-associated peptide; inhibin; activin; vascular endothelial growth factor; integrin; thrombopoietin (TPO); nerve growth factors such as NGF-.beta.; platelet-growth factor; transforming growth factors (TGFs) such as and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines

### Dosages

The skilled artisan is directed to “Remington: The Science and Practice of Pharmacy,” (2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA Office of Biologics standards.

### Screening Phage Libraries by PALM

In certain embodiments, it is desirable to be able to select specific cell types from a heterogeneous sample of an organ or tissue. One method to accomplish such selective sampling is by PALM (Positioning and Ablation with Laser Microbeams). PALM may be used, e.g., to select targeting phage for a particular tissue or cell type.

The PALM Robot-Microbeam uses a precise, computer-guided laser for microablation. A pulsed ultra-violet (UV) laser is interfaced into a microscope and focused through an objective to a beam spot size of less than 1 micrometer in diameter. The principle of laser cutting is a locally restricted ablative photodecomposition process without heating (Hendrix, 1999). The effective laser energy is concentrated on the minute focal spot only and most biological objects are transparent for the applied laser wavelength. This system appears to be the tool of choice for recovery of homogeneous cell populations or even single cells or subcellular structures for subsequent phage recovery. Tissue samples may be retrieved by circumcising a selected zone or a single cell after phage administration to the subject. A clear-cut gap between selected and non-selected area is typically obtained. The isolated tissue specimen can be ejected from the object plane and catapulted directly into the cap of a common micro centrifuge tube in an entirely non-contact manner. The basics of this so called Laser Pressure Catapulting (LPC) method is believed to be the laser pressure force that develops under the specimen, caused by the extremely high photon density of the precisely focused laser microbeam. This tissue harvesting technique allows the phage to survive the microdissection procedure and be rescued.

### Kits

In still further embodiments, the present invention concerns kits for use with the therapeutic and diagnostic methods described above. As the encoded proteins or peptides may be employed to target delivery of a therapeutic to a cell, and/or to detect antibodies or the corresponding antibodies may be employed to detect encoded proteins or peptides, either or both of such components may be provided in the kit. The immunodetection kits will thus comprise, in suitable container means, a protein or peptide or a nucleic acid encoding such, or a first antibody that binds to an encoded protein or peptide, and an immunodetection reagent.

In certain embodiments, the protein or peptide, or the first antibody that binds to the encoded protein or peptide, may be bound to a solid support, such as a column matrix or well of a microtiter plate.

Immunodetection reagents of the kit may take any one of a variety of forms, including those detectable labels that are associated with or linked to the given antibody or antigen, and detectable labels that are associated with or attached to a secondary binding ligand. Exemplary secondary ligands are those secondary antibodies that have binding affinity for the first antibody or antigen, and secondary antibodies that have binding affinity for a human antibody.

Further suitable immunodetection reagents for use in the present kits include the two-component reagent that comprises a secondary antibody that has binding affinity for the first antibody or antigen, along with a third antibody that has binding affinity for the second antibody, the third antibody being linked to a detectable label.

The kits may further comprise a suitably aliquoted composition of the encoded protein or peptide, whether labeled or unlabeled, as may be used to prepare a standard curve for a detection assay.

The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the peptide, peptide conjugate, antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

### I. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Reagents. Cetuximab is a human (h)-mouse (m) chimeric anti-EGFR IgG1 class monoclonal antibody (Goldstein et al., 1995). Monoclonal antibody 528 (isotype IgG2a) and M225 (isotype IgG1) are directed against EGFR. m-IgG and h-IgG were purchased (Sigma). Primary antibodies: Anti-EGFR1 (Tyr1068) and anti-phospho-tyrosine (Cell Signaling) and anti-mouse HRP conjugated (Jackson). Synthetic peptides CVRACGAD, CVRAC, and peptidomimetics _{D}(CARVC) and _{D}(CAAVC) were purchased (PolyPeptide Laboratory). EGFR-derived and unrelated sequences (such as SDNRYIGSW and CEFESC (SEQ ID NO:25)) also served as controls in assays.

Cell Culture and Cell Viability Assays.

Tumor cell lines HN5, UMSCC 10A, GEO and EF43.fgf-4 were culture in standard conditions. Viability was assessed by MTT assays (Sigma) as described (Cardó-Vila et al., 2003). Cells growing in 24-well plates were treated with cetuximab and the peptides or peptidomimetics as indicated for 5 days, washed twice with PBS, incubated in complete media containing MTT (500 μg/ml per well) for 2-4 h, and solubilized with 0.1 N HCl in isopropanol (Cardó-Vila et al., 2003). Samples were read at 570 nm.

Cell Culture.

Tumor cell lines HN5, UMSCC 10A, GEO and EF43.fgf-4 were maintained in high-glucose DMEM, supplemented with 10% heat-inactivated fetal bovine serum, 20 mM HEPES (pH 7.4), 100 IU/ml penicillin, 100 μg/ml streptomycin, and 4 mM glutamine at 5% CO₂ at 37° C. EF43.fgf-4 were grown in G418 as described (Hajitou et al., 2006).

Phage Display Screening and Binding Assays.

Phage peptide screening and binding assays were performed as described (Cardó-Vila et al., 2003). A random phage peptide library displaying the insert CX₇C (where X is any amino acid and C is a cysteine residue) was used for the screening; phage input was 3×10⁹ transducing units (TU). Antibodies, EGF, or TGF-α (R&D Systems) were coated onto microtiter wells as described (Smith and Scott, 1993). Briefly, 10 μg of the indicated antibodies (M225, cetuximab, 528, m-IgG and h-IgG) dissolved in 50 μl PBS were immobilized on microtiter wells overnight at 4° C. Wells were washed twice with PBS, blocked with PBS containing 3% BSA for 1 hr at room temperature (RT), and incubated with the phage library in 50 μl PBS containing 1.5% BSA. After 2 hr at RT, wells were washed ten times with PBS, and phage were recovered by bacterial infection as described (Smith and Scott, 1993; Giordano et al., 2001; Cardó-Vila et al., 2003). Phage recovered on RII (second round), or on RIII for m225 panning, were used for affinity selection on cetuximab; the antibody 528, m-IgG and h-IgG, served as controls. Purification of phage particles and sequencing of phage single-stranded (ss) DNA were performed as described (Arap et al., 1998; Pasqualini et al., 2001). Phage ssDNA from 96 individual clones from each of the second, third, and fourth rounds of selection were prepared, and inserts were sequenced.

Antibodies Against CVRAC Peptide and ELISA.

Rabbits were immunized with KLH-conjugated CVRAC and a purified antibody was obtained. Purification of IgG from rabbit serum produced against the CVRAC peptide (anti-CVRAC) was performed as described (Harlow and Lane, 1999). The capacity of rabbit IgG against CVRAC to recognize CVRAC, _{D}(CARVC), and EGFR was measured by ELISA.

Surface Plasmon Resonance.

SPR was used to determine the inhibitory effect of CVRAC or _{D}(CARVC) on the binding of EGFR to cetuximab on a BIAcore 3000 instrument. SPR was used to determine the inhibitory effect of the peptide CVRAC or the peptidomimetic _{D}(CARVC) on the binding of the EGFR to cetuximab. A capture sensor surface was prepared by covalent immobilization of goat anti-human IgG Fc-specific polyclonal antibody (KPL) approximately 500 resonance units (RU) to a C-1 sensor chip through an NHS/EDC amine coupling kit (Biacore). Binding studies were performed at a flow rate of 10 μl/min at 25° C. by equilibration of the instrument and sensor surface with the running buffer HBS-EP (10 mM HEPES, pH 7.4, 150 mM NaCl, 3 mM EDTA, 0.005% surfactant P-20). Cetuximab, diluted in running buffer (12 μg/ml), was injected over the modified sensor surface with an approximate capture of 25-30 RUs. Samples containing 2.5 ng/ml EGFR in HBS-EP buffer, with or without increasing concentrations of peptides or peptidomimetics, were injected, and binding of EGFR to cetuximab was evaluated. Sensor-chip surfaces were regenerated with 50 mM NaOH. The response obtained on control surfaces (no cetuximab) was subtracted from each binding curve.

Immunoprecipitation and Western Blot Analysis.

Cells were lysed and lysates were used for immunoprecipitation assays as described (Cardó-Vila et al., 2003). Cells were lysed in 20 mM Tris-HCl (pH 7.8), 150 mM NaCl, 1 mM EDTA, 1 mM EGTA, 10 mM DTT, 10 mM PMSF 1% Titron X-100, phosphatase inhibitor I and II (Sigma) and protease inhibitor, sonicated, and clarified by centrifugation. For immunoprecipitation (IP) studies, lysates were incubated with primary antibodies, and the immune complexes were precipitated with protein A-Sepharose beads. Cell lysates or immunoprecipitated proteins were separated by SDS-PAGE, electro-transferred onto nitrocellulose, and probed with primary antibodies and horseradish peroxidase-labeled secondary antibodies.

An anti-phosphotyrosine antibody (PY20) was used for EGFR activation assays. Tumor cells were starved and/or incubated with ligands. After cell lysis, co-IP with phosphorylated EGFR antibody and analysis by SDS-PAGE were performed, followed by electrotransfer to a nitrocellulose filter, the blot was probed with an anti-phosphotyrosine antibody (PY20). Signals were visualized by enhanced chemiluminescence detection (Amersham Biosciences).

Tumor Targeting:

Selective phage homing to tumors was evaluated as described (Hajitou et al., 2006; Arap et al., 1998). Immunocompetent Balb/c female mice bearing EF43.fgf-4-derived breast tumors (Günzburg et al., 1988; Hajitou et al., 2001) were deeply anesthetized and injected intravenously (iv; tail vein) with 10¹⁰ TU of CVRAC-displaying phage, RGD-4C phage (positive control), and CVAAC-displaying or insertless phage (negative controls) in DMEM. Each cohort of mice (n=3 per experiment) with size-matched tumors received a set of test and control phage clones. After 6 h, tumor-bearing mice were perfused through the heart with 20 ml of PBS containing 4% paraformaldehyde (PFA). Tumor and control organs were dissected from each mouse and were placed in PBS containing 30% sucrose for 24 h. Finally, tissues were frozen and sectioned at 5 μm for phage staining as described (Pasqualini et al., 2001). For experimental therapy, Balb/c mice bearing EF43.fgf-4-derived tumors were established, and tumor volumes were determined as described (Arap et al., 1998; Hajitou et al., 2006; Ciardiello et al., 1999). Treatment of tumor-bearing mice started 7 days after cell inoculation (10⁵ cells/mouse).

Immunohistochemistry.

Immunostaining was performed as described (Ozawa et al., 2005). Immunostaining was performed as described (Ozawa et al., 2005). All steps were performed at RT unless stated otherwise. Five micron cryostat sections were air-dried and were subsequently rinsed twice with PBS and once with PBS containing 0.3% Triton X-100 (PBST). Sections were blocked in PBST containing 5% normal goat serum (Jackson ImmunoResearch) for 30 min. Sections were subsequently incubated for 1 h in PBST and 1% normal goat serum containing combinations of the following primary antibodies: Armenian hamster monoclonal anti-mouse CD31 (1:500; Chemicon), or rabbit anti-fd bacteriophage (1:800; Sigma-Aldrich Corp). Sections were rinsed with PBST and were incubated for 1 h in sterile PBST containing appropriate combinations of the following secondary antibodies: goat FITC-conjugated anti-Armenian hamster IgG (1:200; Jackson ImmunoResearch), or goat Cy3-conjugated anti-rabbit IgG (1:400; Jackson ImmunoResearch). Sections were mounted in Vectashield (Vector Laboratories, Inc). Fluorescent images were acquired with an Olympus IX70 inverted fluorescence microscope fitted with an Olympus camera and Magnafire software.

Sequence Alignment.

Peptide-matching software (Mandava et al., 2004) was used to identify motifs resembling targeted ligands. To identify motifs resembling targeted ligands among the selected sequences, the inventors used a peptide-matching software program based on RELIC, an established bioinformatics server for combinatorial peptide analysis and identification of protein-ligand interaction sites (Mandava et al., 2004), designed and implemented through Perl 5.8.1. The program calculates similarity based on a pre-defined amino acid window size (defined empirically and experimentally) between an affinity-selected peptide sequence and the target protein sequence from N-terminus to C-terminus in one-residue shifts to fit the best alignment. The peptide-protein similarity scores for each amino acid residue were calculated based on an amino acid substitution matrix modified to adjust for rare residue representation. In this case, similarity scores were calculated based on a 5-residue window, with every pentamer motif in each selected peptide compared to each pentamer segment of the protein. Empirical similarity score thresholds were set with at least three identical residues plus one similar residue between the peptide and the protein segment.

Statistics.

Student's t-tests were used for statistical analysis of the proliferation assays. For experiments in vivo, statistical significance of the difference was computed by the Kruskal-Wallis test (non-parametric one factor ANOVA method) with P<0.05 for each treatment day. The Wilcoxon Rank Sum test was used to compute differences between each pair-wise study group on a given day of treatment that showed statistical significance from the Kruskal-Wallis test. Statistical analysis was computed by the use of the R-Project for Statistical Computing (v. 2.4.1). Results were considered statistically significant if P<0.05.

### Example 2

### Combinatorial Screening on a Panel of Ligands that Bind to the EGFR

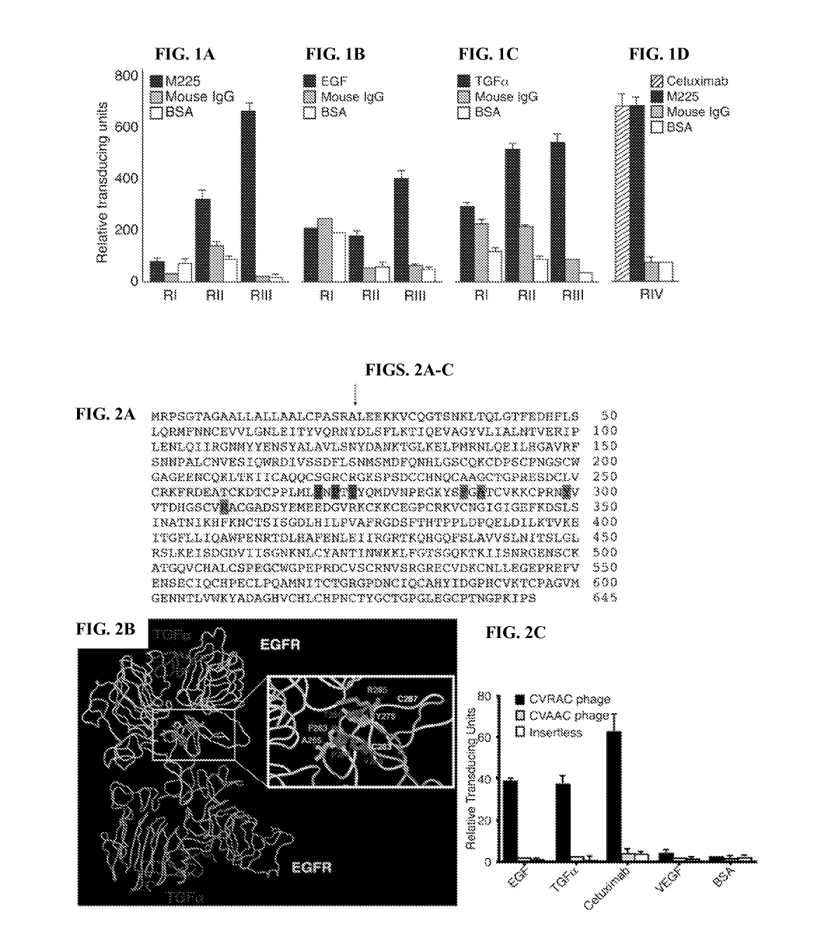

A combinatorial approach was used to identify consensus protein-interacting sites within the EGFR. First, a random library displaying the general peptide arrangement CX₇C on three representative EGFR-ligands [namely; EGF, Tumor Growth Factor alpha (TGFα), and cetuximab] was screened, and phage binding was selected for in consecutive rounds. Serial enrichment in all selections was observed (FIGS. 1A-C). Bovine serum albumin (BSA), VEGF, and irrelevant IgG served as negative controls. As predicted, cetuximab (formerly C225 or IMC225; marketed as Erbitux) showed an overlapping binding profile with its parental murine 225 (M225) version (FIG. 1C and FIG. 1D). After the third round of selection, marked phage binding to each immobilized EGFR ligand was observed, relative to negative controls, as follows: EGF, 8-fold relative to BSA (Student's t-test, P<0.001) and 8-fold relative to VEGF (P<0.001); TGFα, 22-fold relative to BSA (P<0.001), and 15-fold relative to VEGF (P<0.001); M225, 10-fold relative to BSA (P<0.001) and 8-fold relative to irrelevant IgG (P<0.001); and cetuximab, 10-fold relative to BSA (P<0.001) and 8-fold relative to irrelevant IgG (P<0.001).

### Example 3

### Molecular Interaction Between Selected Peptides and the EGFR

A comprehensive protein similarity analysis of selected peptides (n=384) was performed to identify sequences resembling the extracellular domain of the EGFR. Overlapping consensus motifs selected in all three EGFR ligands were identified, mapped, and consolidated within the five dominant candidate regions (Cys227-Cys240, Cys283-Asp290, Cys486-Cys491, Cys547-Cys567, Cys604-Lys618; not accounting for the signal peptide, as indicated) within the primary structure of the receptor (FIG. 2A, yellow highlights). Of note, all such candidate regions contained at least two or more cysteine residues, suggestive of structural motifs.

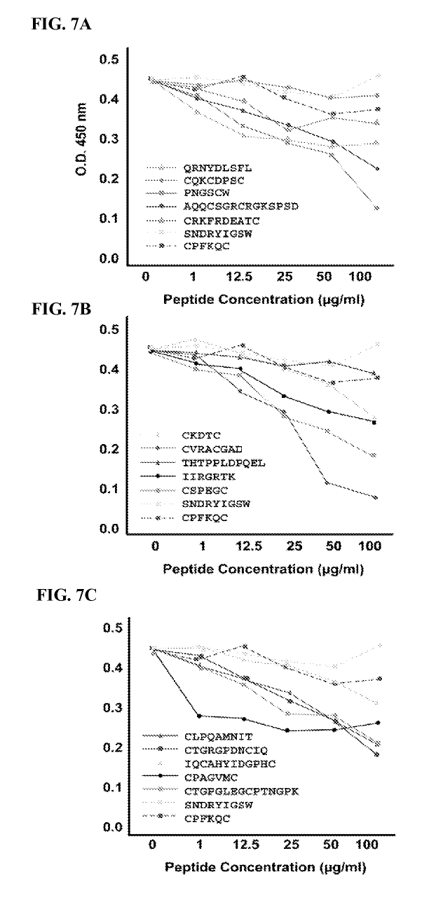

To understand these findings at a protein interactive level, a consensus motif panel (n=15) was generated of synthetic linear and cyclic peptides (Table 1) and used binding to the anti-EGFR monoclonal antibody cetuximab as an initial functional screen (FIGS. 7A-C) to minimize the number of candidate ligands. The inventors have previously expanded this epitope mapping approach to show that selection of random peptide libraries on the repertoire of circulating immunoglobulins from cancer patients (Mintz et al., 2003; Vidal et al., 2004) can identify immunogenic tumor antigens as molecular targets (Arap et al., 2004). Similar methodologies have been applied to therapeutic (Binder et al., 2006; Binder et al., 2007; Riemer et al., 2005) or diagnostic (Jaalouk et al., 2007) antibodies in a strategy that could reveal mechanisms of action (Binder et al., 2007), identify biological reagents for immunization (Riemer et al., 2005), or discover unrecognized antigens (Jaalouk et al., 2007).

The best concentration-dependent ligand peptide in this binding assay (FIGS. 7A-C) was CVRACGAD (residues 283-290), one of the candidate regions encompassing a residue involved in receptor dimerization (Ogiso et al., 2002; Dawson et al., 2005; Dawson et al., 2007) within the EGFR (FIG. 2A and FIG. 2B). Furthermore, binding of the minimized motif CVRAC (residues 283-287) was not significantly different from that of the larger peptide CVRACGAD. Indeed, even the smaller cyclic tripeptide Val-Arg-Ala, containing the residue corresponding to Arg285, was sufficient for binding. In FIG. 2B, light green and light red ribbons indicate the backbone of each EGFR homodimer, and purple designates the TGFα ligand bound to the EGFR (Garrett et al., 2002); the insert details EGFR residues involved in the dimerization site (corresponding to the green and red color coding from FIG. 2A), and the yellow ribbon shows the location and structure of CVRAC within a single chain.

To evaluate whether this motif had selective EGFR-decoy attributes, phage constructs were designed and generated that display the cyclic peptide CVRAC or the corresponding negative control CVAAC, in which Arg has been changed to Ala (through site-directed mutagenesis), and binding to EGFR ligands was measured. The CVRAC-phage preferentially bound to the receptor ligands EGF (17-fold relative to CVAAC-phage, 38-fold relative to insertless phage; Student's t-test, P<0.001), TGFα (13-fold relative CVAAC-phage, 23-fold relative to insertless phage; P<0.001), and cetuximab (23-fold relative CVAAC-phage, 51-fold relative to insertless phage; P<0.001), but not to the negative control proteins VEGF or BSA. A negative control insertless phage (P<0.001) or CVAAC-phage (P<0.001) showed no binding preference (FIG. 2C). These data indicate that the region Cys283-Cys287 of EGFR is implicated in its binding to native ligands and targeted antibodies.

### Example 4

### Short Cyclic Motif as an EGFR-Like Interacting Site

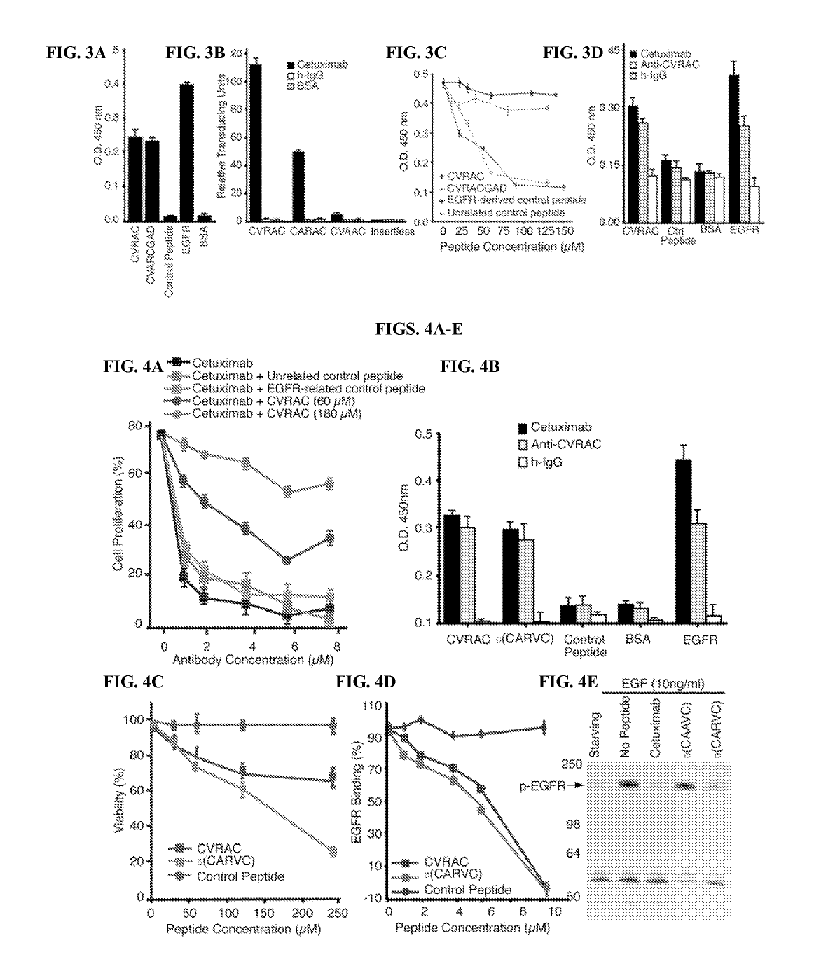

To evaluate CVRAC as a potential drug lead in the development of an EGFR molecular decoy, it was first demonstrated that two synthetic cyclic peptides containing the minimal three-residue cyclic loop CVRAC (i.e., outside a phage display context) bind to cetuximab (FIG. 3A); EGFR served as a positive control and an unrelated peptide as a negative control. Having confirmed that these soluble peptides could recapitulate the EGFR-like binding attributes to a certain extent, the inventors next developed an assay to evaluate the capacity of such peptide ligands to inhibit the binding of cetuximab to the EGFR. By ELISA, the two synthetic peptides, but not two negative control peptides (one with an unrelated sequence and another with an EGFR-derived sequence from region II), blocked the binding of cetuximab to the EGFR in a specific and concentration-dependent manner (FIG. 3B). In both assays (FIG. 3A and FIG. 3B), the binding activity of the synthetic shorter peptide (CVRAC) and the longer peptide (CVRACGAD) were indistinguishable from each other; therefore, the inventors selected the smaller one as a candidate drug lead for derivatization. These data suggest that the interaction of cetuximab with the EGFR is at least partially mediated by CVRAC, a functional, cyclic interacting site embedded within the sequence of the EGFR.

To confirm that the interaction of CVRAC to cetuximab was specific and to identify the residue(s) responsible for peptide binding, phage displaying alanine scanning versions of the peptide were constructed and binding assays to cetuximab or to a negative isotype control were performed (FIG. 3C). CVRAC-displaying phage exhibited marked binding to immobilized cetuximab, in comparison to the negative controls BSA (131-fold; Student's t-test, P<0.001) and isotype antibody (81-fold; P<0.001); moreover, CVRAC-displaying phage bound to a greater extent, relative to negative controls that included insertless phage (96-fold; P<0.001) and CVAAC-displaying phage (48-fold; P<0.001). Consistently, CVAAC-displaying phage lacked binding entirely, but CARAC-displaying phage retained partial activity (˜45% of the CVRAC-displaying phage binding activity), data indicating again that the arginine residue (corresponding to Arg285 within the full-length EGFR) is critical for the interaction of the displayed peptide with cetuximab. Specificity was indicated by the lack of binding to BSA or to the isotype control (FIG. 3C).

The inventors hypothesized that, if the interacting site Cys283-Cys287 within the EGFR exhibits receptor-like properties or biological activity, a synthetic motif might also elicit a cross-reactive humoral immune response. To test this hypothesis, rabbits were immunized with keyhole limpet hemocyanin (KLH) conjugated to the synthetic CVRAC peptide and evaluated the reactivity of purified antibodies by ELISA. Polyclonal antibodies against the soluble motif CVRAC specifically recognized the EGFR (FIG. 3D). These data demonstrate the generation of antibodies against the native receptor and indicate that the interacting site CVRAC within the EGFR behaves as a hapten.

### Example 5

### The Motif CVRAC is Biologically Active

Having established the potential of the EGFR interacting site CVRAC in vitro, the cognate synthetic motif was evaluated in tumor cell lines. The representative colon cancer cell line GEO and the head-and-neck cancer cell line HN5 were chosen because (i) they express the EGFR and represent common human cancers in which EGFR-targeted therapy is used clinically (Gusterson and Hunter, 2009; Jonker et al., 2007; Bonner et al., 2006) and (ii) their respective pattern of tumor response to cetuximab has been established (Posner and Wirth, 2006; Golfinopoulos et al., 2007).

As an experimental baseline, it was confirmed that treatment of these tumor cells in vitro with cetuximab consistently and reproducibly inhibited cell proliferation. To evaluate the biological activity of the synthetic motif CVRAC, either HN5 or GEO tumor cells (FIG. 4A) were incubated with increasing equimolar concentrations of CVRAC or negative control peptides. In both cell lines, a concentration-dependent inhibition by CVRAC of the antibody-dependent inhibition of proliferation was observed, supporting the idea that this was due to the binding to cetuximab as a soluble EGFR decoy. These results demonstrate that CVRAC is biologically active in the context of EGFR-expressing human tumor cells in vitro and is a drug candidate.

### Example 6

### Design, Synthesis, and Development of a Small Drug Decoy

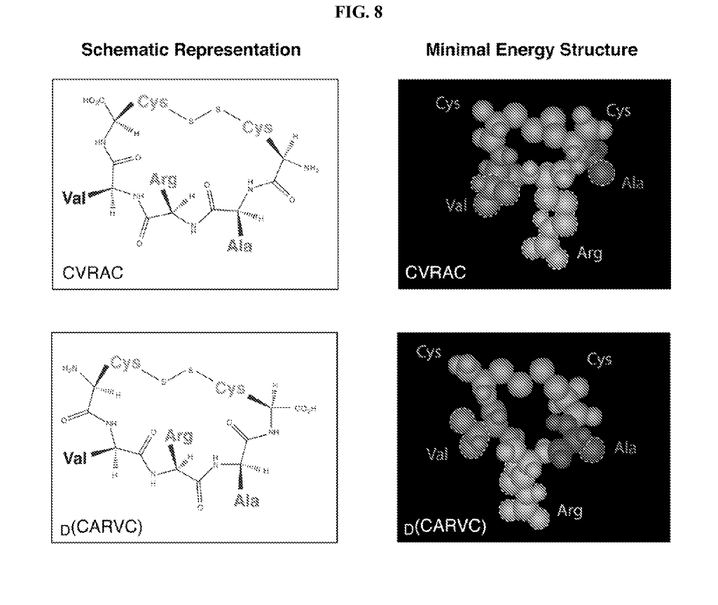

Through solid-phase synthesis, the inventors designed and produced a compound based on the EGFR-interacting site CVRAC (FIG. 8). Because the function of any peptidomimetic-based drug candidate generated through retro-inversion methodology must be empirically validated, the inventors used several assays (FIGS. 4B-D) to determine the activity and biological properties of the retro-inverted drug prototype _{D}(CARVC). The inventors first asked whether antibodies produced against the peptide CVRAC would also recognize _{D}(CARVC) by ELISA (FIG. 4B). _{D}(CARVC) retained binding activity to the antibody cetuximab; in addition, polyclonal anti-CVRAC antibodies recognized both the peptide CVRAC and the drug _{D}(CARVC). The EGFR served as an immobilized positive control, and BSA or control peptides (CVAAC) served as immobilized negative controls. Negative control IgG showed only minimal background binding relative to the specific binding mediated by either anti-CVRAC antibodies or cetuximab (FIG. 4B). This result indicates that antibody recognition of the peptide CVRAC and the drug _{D}(CARVC) is similar in this assay. In summary, the hapten-carrier adduct KLH-CVRAC induces a humoral immune response that recognizes either the peptide CVRAC or the drug _{D}(CARVC) as haptens.

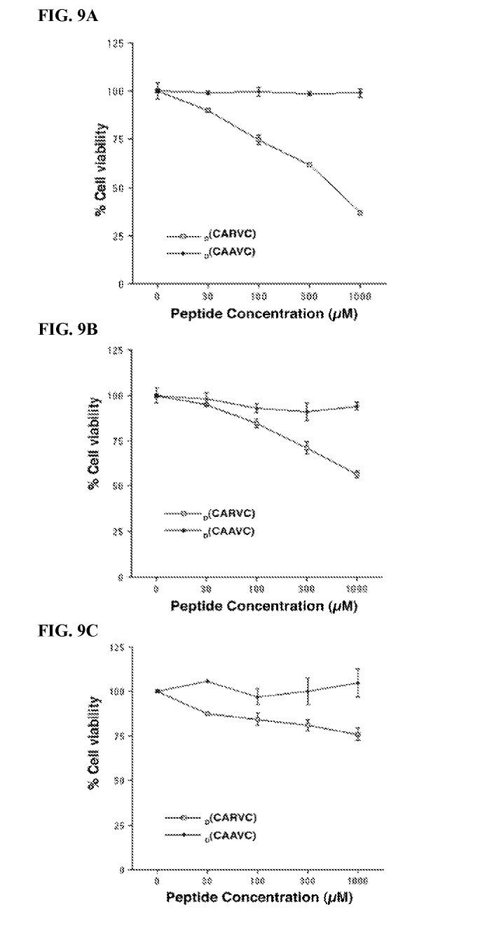

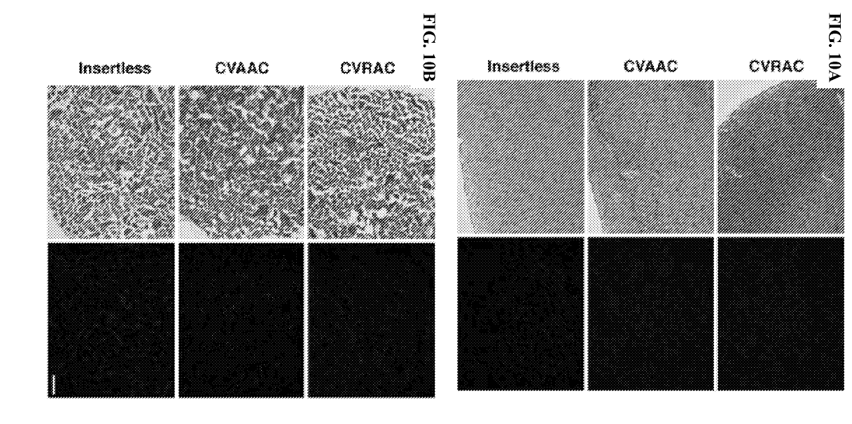

Next, the inventors determined whether the peptide CVRAC or the drug _{D}(CARVC) would affect the proliferation of HN5 cells. Tumor cells exposed to either CVRAC or _{D}(CARVC) proliferated much less in vitro than those exposed to the control peptide (FIG. 4C); this marked effect was specific and concentration-dependent. In addition to HN5 cells, similar results were also observed with GEO cells and with EF43.fgf-4 cells (FIGS. 9A-C). Finally, _{D}(CARVC) activity on an equimolar basis appeared to be more potent, possibly due to the expected proteolytic degradation of the peptide CVRAC in this prolonged (120 h) functional assay.

The capacity of CVRAC and _{D}(CARVC) to block EGFR binding to cetuximab was further assayed by surface plasmon resonance (SPR). An immobilized anti-human Fc monoclonal antibody was used to capture cetuximab; subsequently, the EGFR, either alone or in the presence of the synthetic peptides, was introduced. Both CVRAC and _{D}(CARVC) markedly inhibited the binding of the EGFR to cetuximab (FIG. 4D), relative to the control peptide. The inhibition was concentration-dependent, with an IC₅₀ value of ˜5.4 mM for CVRAC and ˜4.8 mM for _{D}(CARVC); the observed similar activity of the two agents reflects the lack of enzymatic activity in this serum-free assay. No binding inhibition was observed with control peptides at equimolar concentrations (FIG. 4D). Low-affinity interactions (micromolar range) were observed with both agents by nuclear magnetic resonance (NMR)-spectroscopy.

Finally, to determine whether EGFR activation was inhibited after treatment with the drug candidate, the inventors incubated tumor cells with synthetic peptides, _{D}(CARVC), or cetuximab in the presence or absence of EGF. Treatment of tumor cells with EGF led to the tyrosine phosphorylation of a specific 170-kDa protein (FIG. 4E); as expected, no phosphorylation was observed in non-EGFR expressing control cells. Both, cetuximab and _{D}(CARVC)—but not the negative control drug [_{D}(CAAVC), a synthetic peptidomimetic with a mutation corresponding to EGFR Arg285Ala]—effectively inhibited the EGF-induced tyrosine phosphorylation in this functional assay (FIG. 4E), thus suggesting a new EGFR inhibitory mechanism. To dissect the downstream signal transduction cascade in this setting, proliferative, survival, and migratory pathways were examined. In experiments with human HN5 head and neck cells, _{D}(CARVC) was observed to differentially inhibit EGF-induced phosphorylation of ERK and AKT but not p38.

### Example 7

### Tumor Targeting and Pre-Clinical Validation of _{D}(CARVC)

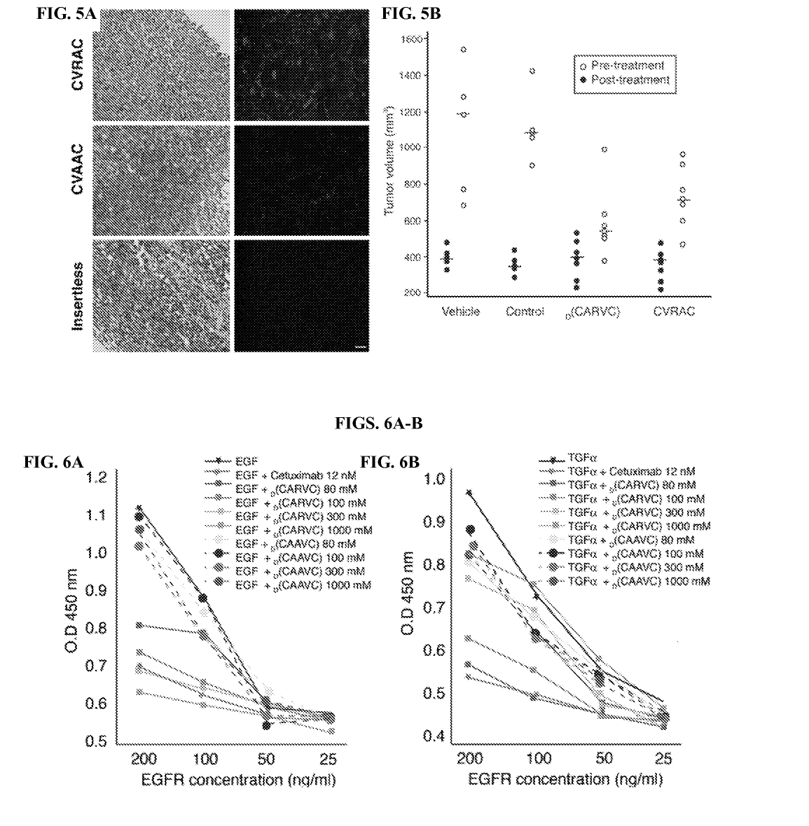

Having demonstrated the biological activity of these EGFR-derived agents in vitro, their potential for use in vivo was next evaluated (FIGS. 5A-B). To behave as EGFR-decoys, the inventors hypothesized that these agents may home to an EGFR “ligand-rich”tumor microenvironment (i.e., with high local concentrations of the native ligands EGF and/or TGFα). Therefore, the capacity of CVRAC-displaying phage to target tumors in vivo was determined by administration of CVRAC-displaying phage or controls (either CVAAC-displaying phage or insertless control phage) intravenously (i.v.) into immunocompetent (Balb/c) female mice bearing mammary tumors (FIG. 5A). The inventors chose to test a standard tumor model in which EF43.fgf-4 cells are administered subcutaneously (s.c.) to induce very rapid growth of highly vascularized tumors in immunocompetent mice (Hajitou et al., 2006). In histological sections of fixed tissue, there was marked staining of the tumors in mice receiving CVRAC-displaying phage but only background staining in control organs. In contrast, negative control phage (either CVAAC-displaying phage or insertless phage) were not detected in tumors (FIG. 5A) or in control organs (FIGS. 10A-B). The inventors used the same isogenic tumor model (Hajitou et al., 2006) to evaluate whether the peptide CVRAC or the drug prototype _{D}(CARVC) could suppress tumor growth in vivo. Tumor-bearing mice received vehicle alone, the peptide CVRAC, the drug _{D}(CARVC), or control peptides (FIG. 5B). The inventors observed differences in tumor growth as early as 5 days after treatment. At the end of two weeks, mice treated with _{D}(CARVC) exhibited significantly smaller tumor volumes (550±50 mm³, P=0.02), relative to tumor-bearing mice that received negative control peptide (1,120±120 mm³; t-test). Tumors in mice treated with control peptide behaved similarly to tumors in mice receiving vehicle alone (1,200±135 mm³), data indicating that a control peptide had no measurable effect. The CVRAC peptide also showed therapeutic efficacy in vivo but, because of enzymatic degradation, the tumor responses observed were somewhat inferior to those of _{D}(CARVC). These results confirm that ligand-directed viral particles and EGFR-derived peptidomimetics target tumors. Consistent with an EGFR-decoy activity, these results likely represent homing due to high concentrations of EGFR ligands in the tumor microenvironment in vivo. It has long been established that D-amino acid oxidase is the only mammalian enzyme that catabolize D-peptidomimetics in the kidney (Meister, 1965); without wishing to be bound by any theory, it is anticipated that the drug excretion mechanism may be renal.

### Example 8

### Mechanism of Action as a Candidate Drug Decoy

Given the promising results observed in vivo, the mechanism of action of _{D}(CARVC) as an EGFR-targeted soluble decoy were further investigated. Decreasing molar concentrations of EGFR were immobilized in vitro, after which the native ligand (EGF or TGFα) was used to establish the baseline for the binding of EGF to the EGFR, or of TGFα to the EGFR. As predicted, cetuximab displaced EGF at low nanomolar concentrations and thus served as a positive control. The drug _{D}(CARVC) displaced EGF and yielded a concentration-dependent effect (from 30 to 1,000 mM). Moreover, the magnitude of ligand displacement elicited by _{D}(CARVC) at 300 mM was similar to that of cetuximab in this assay (FIG. 6A). Finally, concentration-dependent displacement of TGFα by _{D}(CARVC) was observed (FIG. 6B). Without wishing to be bound by any theory, these data support the EGFR-decoy effect as a mechanism of action of this prototype.

Li et al. (2005) have reported that cetuximab interacts with domain III of the soluble extracellular region of the EGFR. Notably, the EGFR-ligand peptide CVRAC, is on domain II. The peptides presented here may be used as molecular decoys for EGF- and TGF α-related pathways.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 4,472,509
U.S. Pat. No. 5,021,236
U.S. Pat. No. 5,206,347
U.S. Pat. No. 5,401,511
U.S. Pat. No. 5,603,872
U.S. Pat. No. 5,889,155
Arap et al., Cancer Cell, 6:275-284, 2004.
Arap et al., Curr. Opin. Oncol., 10:560-565, 1998a.
Arap et al., Science, 279:377-380, 1998b.
Baba et al., Cancer Res., 60:6886-6889, 2000.
Baichwal and Sugden, In: Gene Transfer, Kucherlapati (Ed.), Plenum Press, NY, 117-148, 1986.
Bakhshi et al., Cell, 41(3):899-906, 1985.
Barany and Merrifield, In: The Peptides, Gross and Meienhofer (Eds.), Academic Press, NY, 1-284, 1979.
Baselga, Science, 312:1175-1178, 2006.
Binder et al., Blood, 108:1975-1980, 2006.
Binder et al., Cancer Res., 67:3518-3523, 2007.
Bonner et al., N. Engl. J. Med., 354:567-578, 2006.
Cardó-Vila et al., Mol. Cell, 11:1151-1162, 2003.
Chen and Okayama, Mol. Cell Biol., 7(8):2745-2752, 1987.
Chorev and Goodman, Acc. Chem. Res., 26:266-273, 1993.
Ciardiello et al., Clin. Cancer Res., 5:909-916, 1999.
Cleary and Sklar, Proc. Natl. Acad. Sci. USA, 82(21):7439-7443, 1985.
Coffin, In: Virology, Fields et al. (Eds.), Raven Press, NY, 1437-1500, 1990.
Coupar et al., Gene, 68:1-10, 1988.
Dassonville et al., Crit. Rev. Oncol. Hematol., 62:53-61, 2007.
Dawson et al., Mol. Cell Biol., 25:7734-7742, 2005.
Dawson et al., Structure, 15:942-954, 2007.
Dlugosz et al., Cancer Res., 55:1883-1893, 1995.
Eliceiri and Cheresh, Curr. Opin. Cell. Biol., 13:563-568, 2001.
Ellerby et al. Nature Med., 9:1032-1038, 1999.
Folkman, In: Cancer: Principles and Practice, eds. DeVita et al., 3075-3085, Lippincott-Raven, NY, 1997.
Friedmann, Science, 244:1275-1281, 1989.
Fujimoto et al., Cancer Res., 65:11478-11485, 2005.
Garrett et al., Cell, 110:763-773, 2002.
Gilmore and Riese, Oncol. Res., 14:589-602, 2004.
Giordano et al., Nat. Med., 7:1249-1253, 2001.
Goldstein et al., Clin. Cancer Res., 1:1311-1318, 1995.
Golfinopoulos et al., Lancet. Oncol., 8:898-911, 2007.
Gomez-Foix et al., J. Biol. Chem., 267:25129-25134, 1992.
Goodman & Gilman's “The Pharmacological Basis of Therapeutics”
Gopal, Mol. Cell Biol., 5:1188-1190, 1985.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al., J. Gen. Virl., 36(1):59-74, 1977.
Grunhaus and Horwitz, Seminar in Virology, 3:237-252, 1992.
Günzburg et al., Carcinogenesis, 9:1849-1856, 1988.
Gusterson and Hunter, Lancet. Oncol., 10:522-527, 2009.
Hajitou et al., Cancer Res., 61:3450-3457, 2001.
Hajitou et al., Cell, 125:385-398, 2006.
Harlow and Lane, In: Using Antibodies: A Laboratory Manual, Cold Spring Harbor Laboratory, Cold Spring Harbor, N.Y., 74-82, 1999.
Harlow and Lane, In: Antibodies: A Laboratory Manual, Cold Spring Harbor Laboratory, Cold Spring Harbor, N.Y., 346-348, 1988.
Hendrix, Current Biol., 9:914-917, 1999.
Hermonat and Muzycska, Proc. Natl. Acad. Sci. USA, 81:6466-6470, 1984.
Herz and Gerard, Proc. Natl. Acad. Sci. USA, 90:2812-2816, 1993.
Horwich et al. J. Virol., 64:642-650, 1990.
Hubbard, Cancer Cell, 7:287-288, 2005.
Jaalouk et al., Cancer Res., 67:9623-9629, 2007.
James et al., Science, 299:1362-1367, 2003.
Johnson et al., In: Biotechnology And Pharmacy, Pezzuto et al. (Eds.), Chapman and Hall, NY, 1993.
Jones and Shenk, Cell, 13:181-188, 1978.
Jonker et al., N. Engl. J. Med., 357:2040-2048, 2007.
Karapetis et al., N. Engl. J. Med., 359:1757-1765, 2008.
Kawamoto et al., Proc. Natl. Acad. Sci. USA, 80:1337-1341, 1983.
Kerr et al., Br. J. Cancer, 26(4):239-257, 1972.
Klein et al., Nature, 430:1040-1044, 2004.
Klein et al., Nature, 453:1271-1275, 2008.
Le Gal La Salle et al., Science, 259:988-990, 1993.
Levrero et al., Gene, 101:195-202, 1991.
Li et al., Cancer Cell, 7:301-311, 2005.
Lynch et al., N. Engl. J. Med., 350:2129-2139, 2004.
Mandava et al., Proteomics, 4:1439-1460, 2004.
Mann et al., Cell, 33:153-159, 1983.
Meister, In: Biochemistry of the Amino Acids, 2^{nd} Ed., 1:364, Academic Press, NY, 1965.
Mellinghoff et al., N. Engl. J. Med., 353:2012-2024, 2005.
Mendelsohn and Baselga, Sem. Oncol., 33:369-385, 2006.
Merrifield, Science, 232(4748):341-347, 1986.
Mintz et al., Nat. Biotechnol., 21:57-63, 2003.
Nicolas and Rubinstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176, 1987.
Ogiso et al., Cell, 110:775-787, 2002.
Ozawa et al., Cancer, 104:2104-2115, 2005.
Paez et al., Science, 304:1497-1500, 2004.
Paskind et al., Virology, 67:242-248, 1975.
Pasqualini et al., In: Phage Display. A Laboratory Manual, Barbas et al. (Eds.), Cold Spring Harbor Laboratory Press, NY, 22: 1-24, 2001.
Physicians Desk Reference
Posner and Wirth, N. Engl. J. Med., 354:634-636, 2006.
Potter et al., Proc. Natl. Acad. Sci. USA, 81:7161-7165, 1984.
Racher et al., Biotech. Tech., 9:169-174, 1995.
Ragot et al., Nature, 361:647-650, 1993.
Remington: The Science and Practice of Pharmacy, 20th Ed., Gennaro, Lippincott, 2000.
Rich et al., Hum. Gene Ther., 4:461-476, 1993.
Ridgeway, In: Vectors: A Survey of Molecular Cloning Vectors and Their Uses, Rodriguez et al. (Eds.), Stoneham: Butterworth, 467-492, 1988.
Riemer et al., J. Nat. Cancer Inst., 97:1663-1670, 2005.
Rini et al., Science, 255:959-965, 1992.
Rippe, et al., Mol. Cell Biol., 10:689-695, 1990.
Rosenfeld et al., Science, 252:431-434, 1991.
Rosenfeld, et al., Cell, 68:143-155, 1992.
Scott et al., Proc. Natl. Acad. Sci. USA, 104:4071-4076, 2007.
Sharma et al., Nat. Rev. Cancer, 7:169-181, 2007.
Smith and Scott, Methods Enzymol., 217:228-257, 1993.
Stevens et al., Proc. Natl. Acad. Sci. USA, 85:6895-6899, 1988.
Stewart and Young, In: Solid Phase Peptide Synthesis, 2d. ed., Pierce Chemical Co., 1984.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Sweet-Cordero et al., Nat. Genet., 37:48-55, 2004.
Tam et al., J. Am. Chem. Soc., 105:6442, 1983.
Temin, In: Gene Transfer, Kucherlapati (Ed.), NY, Plenum Press, 149-188, 1986.
Tsujimoto et al., Nature, 315:340-343, 1985.
Tur-Kaspa et al., Mol. Cell Biol., 6:716-718, 1986.
Vidal et al., Oncogene, 23:8859-8867, 2004.
Wong et al., Gene, 10:87-94, 1980.
Wu and Wu, Biochemistry, 27: 887-892, 1988.
Wu and Wu, J. Biol. Chem., 262:4429-4432, 1987.

1. A peptide comprising _{D}(ARV) or VRA, wherein the peptide is 50 or less amino acids in length, and wherein the peptide can selectively bind epidermal growth factor (EGF) or transforming growth factor alpha (TGF-α).

2. The peptide of claim 1, wherein the peptide is 15 or less amino acids in length.

3. The peptide of claim 2, wherein the peptide is 10 or less amino acids in length.

4. The peptide of claim 1, wherein the peptide is a cyclic peptide.

5. The peptide of claim 1, wherein the peptide comprises _{D}(CARVC) (SEQ ID NO:1) or CVRAC (SEQ ID NO:2).

6. The peptide of claim 5, wherein the peptide comprises _{D}(CARVC), and wherein the peptide is 7 or less amino acids in length.

7. The peptide of claim 6, wherein the peptide consists of _{D}(CARVC).

8. The peptide of claim 1, wherein the peptide is 7 amino acids or less in length.

9. The peptide of claim 1, wherein the peptide comprises CVRAC.

10. The peptide of claim 1, wherein the peptide is conjugated or fused to a second agent.

11. The peptide of claim 10, wherein the second agent is a polypeptide.

12. The peptide of claim 11, prepared by a process comprising obtaining a nucleic acid coding region the encodes the peptide and fusing said coding region in frame to a nucleic acid coding region for the polypeptide to form a fused coding region, and expressing said fused coding regions to provide the peptide fused with said polypeptide.

13. The peptide of claim 10, wherein the second agent is a therapeutic or diagnostic agent.

14. The peptide of claim 13, wherein the second agent is a therapeutic agent, further defined as a drug, a chemotherapeutic agent, a radioisotope, a pro-apoptosis agent, an anti-angiogenic agent, a hormone, a cytokine, a cytotoxic agent, a cytocidal agent, a cytostatic agent, a peptide, a protein, an antibiotic, an antibody, a Fab fragment of an antibody, a hormone antagonist, a nucleic acid or an antigen.

15. The peptide of claim 14, wherein the second agent is an anti-angiogenic agent selected from the group consisting of thrombospondin, angiostatin, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4, and minocycline.

16. The peptide of claim 14, wherein the second agent is a pro-apoptosis agent selected from the group consisting of etoposide, ceramide sphingomyelin, Bax, Bid, Bik, Bad, caspase-3, caspase-8, caspase-9, fas, fas ligand, fadd, fap-1, tradd, faf, rip, reaper, apoptin, interleukin-2 converting enzyme or annexin V.

17. The peptide of claim 14, wherein the second agent is a cytokine selected from the group consisting of interleukin 1 (IL-1), IL-2, IL-5, IL-10, IL-12, IL-18, interferon-γ (IF-γ), IFα, IF-β, tumor necrosis factor-α (TNF-α), or GM-CSF (granulocyte macrophage colony stimulating factor).

18. The peptide of claim 13, wherein the second agent is a molecular complex.

19. The peptide of claim 18, wherein the complex is a virus, a bacteriophage, a bacterium, a liposome, a microparticle, a magnetic bead, a yeast cell, a mammalian cell or a cell.

20. The peptide of claim 19, wherein the complex is a virus or a bacteriophage.

21. The peptide of claim 20, wherein the virus is chosen from the group consisting of adenovirus, retrovirus adeno-associated virus (AAV), and AAVP.

22. The peptide of claim 20, wherein the virus is further defined as containing a gene therapy vector.

23. The peptide of claim 19, wherein the peptide is attached to a eukaryotic expression vector.

24. The peptide of claim 23, wherein the vector is a gene therapy vector.

25. The peptide of claim 13, wherein the second agent is a diagnostic agent.

26. The method of claim 25, wherein the diagnostic agent is an imaging agent.

27. The method of claim 26, wherein the imaging agent comprises chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) erbium (III), lanthanum (III), gold (III), lead (II), or bismuth (III).

28. The method of claim 26, wherein the agent comprises a radioisotope, and the radioisotope is astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} or yttrium⁹⁰.

29. The peptide of claim 1, wherein the peptide is comprised in a pharmaceutically acceptable composition.

30. A method of making a polypeptide in accordance with claim 13, comprising obtaining a nucleic acid coding region that encodes the peptide and fusing said coding region in frame to a nucleic acid coding region for the polypeptide to form a fused coding region, and expressing said fused coding regions to provide the peptide fused with said polypeptide.

31. A nucleic acid that encodes a protein or peptide comprising _{D}(ARV) or VRA; wherein the peptide is 10 or less amino acids in length.

32. The nucleic acid of claim 31, wherein the peptide comprises _{D}(CARVC) or CVRAC.

33. The nucleic acid of claim 31, wherein the nucleic acid is operably linked to a heterologous promoter.

34. A method of treating cancer comprising administering to a subject the peptide of claim 1.

35. The method of claim 22, wherein the cancer is selected from the group consisting of lung cancer, gastrointestinal cancer, colon cancer, anal cancer, and glioblastoma multiforme.

36. The method of claim 34, wherein the subject is a mammal.

37. The method of claim 36, wherein the mammal is a human.

38. The method of claim 37, wherein the peptide is administered in a pharmaceutically acceptable carrier.

39. The method of claim 34, further comprising administering a second therapeutic agent to the subject.

40. A method for imaging cells expressing epidermal growth factor (EGF) or transforming growth factor alpha (TGF-α) comprising exposing cells to the peptide of claim 26.

41. The method of claim 40, wherein the cells comprise cancer cells.

42. The method of claim 41, wherein the cancer cells comprise lung cancer cells, gastrointestinal cancer cells, anal cancer cells, or glioblastoma multiforme cells.


SEQUENCE LISTING (25 sequences)

SEQ ID NO: 1 - protein, 5 aa, Artificial Sequence
  Synthetic peptide
CARVC

SEQ ID NO: 2 - protein, 5 aa, Artificial Sequence
  Synthetic peptide
CVRAC

SEQ ID NO: 3 - protein, 8 aa, Artificial Sequence
  Synthetic peptide
CVRACGAD

SEQ ID NO: 4 - protein, 9 aa, Artificial Sequence
  Synthetic peptide
SDNRYIGSW

SEQ ID NO: 5 - protein, 8 aa, Artificial Sequence
  Synthetic peptide
CQKCDPSC

SEQ ID NO: 6 - protein, 5 aa, Artificial Sequence
  Synthetic peptide
CARAC

SEQ ID NO: 7 - protein, 5 aa, Artificial Sequence
  Synthetic peptide
CVAAC

SEQ ID NO: 8 - protein, 5 aa, Artificial Sequence
  Synthetic peptide
CAAVC

SEQ ID NO: 9 - protein, 9 aa, Artificial Sequence
  Synthetic peptide
QRNYDLSFL

SEQ ID NO: 10 - protein, 8 aa, Artificial Sequence
  Synthetic peptide
CQKCDPSC

SEQ ID NO: 11 - protein, 6 aa, Artificial Sequence
  Synthetic peptide
PNGSCW

SEQ ID NO: 12 - protein, 15 aa, Artificial Sequence
  Synthetic peptide
AQQCSGRCRGKSPSD

SEQ ID NO: 13 - protein, 10 aa, Artificial Sequence
  Synthetic peptide
CRKFRDEATC

SEQ ID NO: 14 - protein, 5 aa, Artificial Sequence
  Synthetic peptide
CKDTC

SEQ ID NO: 15 - protein, 8 aa, Artificial Sequence
  Synthetic peptide
CVRACGAD

SEQ ID NO: 16 - protein, 11 aa, Artificial Sequence
  Synthetic peptide
THTPPLDPQEL

SEQ ID NO: 17 - protein, 7 aa, Artificial Sequence
  Synthetic peptide
IIRGRTK

SEQ ID NO: 18 - protein, 6 aa, Artificial Sequence
  Synthetic peptide
CSPEGC

SEQ ID NO: 19 - protein, 9 aa, Artificial Sequence
  Synthetic peptide
CLPQAMNIT

SEQ ID NO: 20 - protein, 11 aa, Artificial Sequence
  Synthetic peptide
CTGRGPDNCIQ

SEQ ID NO: 21 - protein, 12 aa, Artificial Sequence
  Synthetic peptide
IQCAHYIDGPHC

SEQ ID NO: 22 - protein, 6 aa, Artificial Sequence
  Synthetic peptide
CPAGVM

SEQ ID NO: 23 - protein, 15 aa, Artificial Sequence
  Synthetic peptide
CTGPGLEGCPTNGPK

SEQ ID NO: 24 - protein, 14 aa, Artificial Sequence
  Synthetic peptide
KLAKLAKKLAKLAK

SEQ ID NO: 25 - protein, 6 aa, Artificial Sequence
  Synthetic peptide
CEFESC